(19) United States
(12) Patent Application Publication
(10) Pub. No.: US 2022/0172849 A1
(43) Pub. Date: Jun. 2, 2022

(54) System and Techniques for Clinical Documentation and Editing

(71) Applicant: 3M INNOVATIVE PROPERTIES COMPANY, St. Paul, MN (US)

(72) Inventors: David R. Bacon, Sandy, UT (US); Linda L. McIntyre, Cincinnati, OH (US); Donna C. Smith, Roswell, GA (US); Benjamin M. Templeton, Eagle Mountain, UT (US); Richard H. Wolniewicz, Longmont, CO (US)

(21) Appl. No.: 17/651,580

(22) Filed: Feb. 18, 2022

Related U.S. Application Data
(63) Continuation-in-part of application No. 15/887,385, filed on Feb. 2, 2018.
Application No. 15/887,385 is a continuation of application No. 13/416,527, filed on Mar. 9, 2012, now abandoned.
(60) Provisional application No. 61/539,410, filed on Sep. 26, 2011.

(51) Int. Cl. G16H 70/60; G06Q 10/06; G16H 10/60; G06Q 10/10; G16H 15/00

(52) U.S. Cl. CPC G16H 70/60; G06Q 10/06; G16H 15/00; G06Q 10/10; G16H 10/60

(57) ABSTRACT

In one example, this disclosure describes a method of processing medical data via one or more computers. The method may comprise identifying a medical code within a medical record, and identifying whether the medical code is specified or unspecified. If the medical code is specified, editing may be avoided without generating any query for further input by a physician. If the medical code is unspecified, the method further includes determining whether a suppression code appears in the medical record. If a suppression code appears, editing may be avoided without generating any query for further input by the physician. However, if a suppression code does not appear, the method further includes searching for key terms in the medical record. If key terms are present in the medical record, a query may be generated for the physician for additional clarification.

This application is a continuation-in-part of U.S. application Ser. No. 15/887,385, filed Feb. 2, 2018, pending, which is a continuation of U.S. application Ser. No. 13/416,527 filed Mar. 9, 2012, abandoned, which claims the benefit of U.S. Provisional Application 61/539,410, filed Sep. 26, 2011, the entire content of which is incorporated herein by reference.

### TECHNICAL FIELD

This disclosure relates to healthcare at medical facilities and to the documentation and editing of medical records.

### BACKGROUND

In the medical field, clinical documentation is of paramount importance for monitoring patient well being and accurately representing medical conditions to insurance companies, governmental agencies, or other payers. The emergence and use of electronic medical records and electronic documentation can help clinical documentation processes, but may present many challenges.

### SUMMARY

This disclosure describes systems and techniques for processing medical data via one or more computers. The systems and techniques may be used by a medical reviewer (sometimes referred to as a “documentation specialist”). The techniques and systems described herein can help to automate (or partially automate) the coding process associated with medical record review. In this manner, the process can be improved and/or simplified. The techniques may apply one or more rules to define when documentation for medical records is sufficient and when further review of the medical records is needed. The rules may define when the documentation specialist should review the medical records and may automate the process by avoiding the display of medical records to the documentation specialist when the documentation in the medical records is sufficient. The rules may further define when physician review is needed, and may automate the process by avoiding physician review when the documentation in the medical records is sufficient or when review by the documentation specialist may suffice prior to (and possibly in lieu of) physician review. Machine learning techniques are also described which may be used in conjunction with, or in lieu of, one or more of the rules.

In one example, this disclosure describes a method of processing medical data via one or more computers. The method comprises identifying, via the one or more computers, a medical code within a medical record, and identifying, via the one or more computers, whether the medical code is one of a plurality of specified medical codes or one of a plurality of unspecified medical codes, wherein the specified medical codes are sufficient to reflect and accurately represent a medical condition to a payer and the unspecified medical codes are defined as requiring additional information for medical condition clarity. If the medical code is one of the specified medical codes, the method includes avoiding display, via the one or more computers, of clinical edit options for the medical record without generating a query for further input by a physician. Wherein the clinical edit options are used by a documentation specialist to determine whether a query for further input by a physician should be generated and allow a documentation specialist to edit one or more aspects of the medical record. If the medical code is one of the unspecified medical codes, the method includes determining, via the one or more computers, whether one of a plurality of suppression codes associated with the medical code appears in the medical record. If one of the suppression codes appears in the medical record, the method includes avoiding display, via the one or more computers of the clinical edit options for the medical record without generating the query for further input by the physician. If one of the suppression codes does not appear in the medical record, the method includes searching for one or more key terms in the medical record via the one or more computers. If the one or more key terms exist in the medical record, the method includes causing display of the clinical edit options for the medical record via the one or more computers. If one or more key terms are present in the medical record, the method includes determining whether or not to generate a query for further input by the physician via the one or more computers. The documentation specialist, based upon displayed clinical edit options, determines whether or not a clinical edit warrants a physician query as only a physician is authorized to directly edit medical documentation. In essence, clinical edit options are displayed to provide clarity and enable documentation specialists to generate queries when documentation is insufficient to reflect and accurately represent a medical condition. For some queries, the determination by the documentation may be automated through statistical machine learning techniques where the absence of suppression codes and presence of key terms from the medical record have a high probability of generating a particular query.

In another example, this disclosure describes a computerized system for processing medical data, the system comprising a computer that includes a processor and a memory, wherein the processor is configured to include an editing module. The editing module identifies a medical code within a medical record stored in the memory, and identifies whether the medical code is one of a plurality of specified medical codes or one of a plurality of unspecified medical codes, wherein the specified medical codes are sufficient to reflect and accurately represent a medical condition to a payer and the unspecified medical codes are defined as requiring additional information for medical condition clarity. If the medical code is one of the specified medical codes, the editing module avoids causing display of clinical edit options for the medical record without generating a query for further input by a physician. If the medical code is one of the unspecified medical codes, the editing module determines whether one of a plurality of suppression codes associated with the medical code appears in the medical record stored in the memory. If one of the suppression codes appears in the medical record the editing module avoids causing display of the clinical edit options for the medical record without generating the query for further input by the physician. If one of the suppression codes does not appear in the medical record, the editing module searches for one or more key terms in the medical record stored in the memory. If the one or more key terms exist in the medical record, the editing module causes display of the clinical edit options for the medical record stored in the memory. It will be apparent to one of skill in the art that clinical edit options may be displayed or be stored in memory to be accessed and viewed at a later time. If one or more key terms are present in the medical record, the editing module determines whether or not to generate a query for further input by the physician.

In another example, this disclosure describes a device for processing medical data. In this example, the device comprises means for identifying a medical code within a medical record, and means for identifying whether the medical code is one of a plurality of specified medical codes or one of a plurality of unspecified medical codes, wherein the specified medical codes are sufficient to reflect and accurately represent a medical condition to a payer and the unspecified medical codes are defined as requiring additional information for medical condition clarity. If the medical code is one of the specified medical codes, the device comprises means for avoiding display of clinical edit options for the medical record without generating a query for further input by a physician. If the medical code is one of the unspecified medical codes, the device comprises means for determining whether one of a plurality of suppression codes associated with the medical code appears in the medical record. If one of the suppression codes appears in the medical record, the device comprises means for avoiding display of the clinical edit options for the medical record without generating the query for further input by the physician. If one of the suppression codes does not appear in the medical record, the device comprises means for searching for one or more key terms in the medical record. If one or more key terms are present in the medical record, the device comprises means for causing display of the clinical edit options for the medical record. If the one or more key terms are present in or absent from the medical record, the device comprises means for generating the query for further input by the physician.

The techniques of this disclosure may be implemented at least partially in hardware, such as a processor or discrete logic circuits. The techniques may also be implemented using aspects of software or firmware in combination with the hardware. If implemented at least partially in software or firmware, the software or firmware may be executed in one or more hardware processors, such as a microprocessor, application specific integrated circuit (ASIC), field programmable gate array (FPGA), or digital signal processor (DSP). The software that executes the techniques may be initially stored in a computer-readable storage medium and loaded and executed in the processor. The processor may execute modules to perform the techniques of this disclosure, and the modules may comprise combinations of software and hardware, e.g., software routines executing on the processor.

Accordingly, this disclosure also contemplates a computer-readable storage medium comprising instructions that when executed in a processor cause the processor to process medical data, wherein upon execution the instructions cause the processor to identify a medical code within a medical record, and identify whether the medical code is one of a plurality of specified medical codes or one of a plurality of unspecified medical codes, wherein the specified medical codes are sufficient to reflect and accurately represent a medical condition to a payer and the unspecified medical codes are defined as requiring additional information for medical condition clarity. If the medical code is one of the specified medical codes, the instructions cause the processor to avoid display of clinical edit options for the medical record without generating a query for further input by a physician. If the medical code is one of the unspecified medical codes, the instructions cause the processor to determine whether one of a plurality of suppression codes associated with the medical code appears in the medical record. If one of the suppression codes appears in the medical record, the instructions cause the processor to avoid display of the clinical edit options for the medical record without generating the query for further input by the physician. If one of the suppression codes does not appear in the medical record, the instructions cause the processor to search for one or more key terms in the medical record. If one or more key terms are present in the medical record, the instructions cause the processor to cause display of the clinical edit options for the medical record. If one or more key terms are present in the medical record, the instructions cause the processor to generate the query for further input by the physician.

In other examples, this disclosure describes hybrid techniques that use fixed or pre-defined rules in conjunction with adaptive rules that can change based on statistical machine learning. For example, this disclosure describes a method of processing medical data via one or more computers. The method may comprise parsing a medical record via the one or more computers, determining a first outcome for coding the medical record based on one or more pre-defined rules, determining a second outcome for coding the medical record based on one or more adaptive rules, wherein the adaptive rules are defined based on statistical machine learning based on processing of other medical records, selecting between the first and second outcomes, and causing output related to coding the medical record based on the selected outcome.

In another example, this disclosure describes a computerized system for processing medical data, the system comprising a computer that includes a processor and a memory, wherein the processor is configured to include an editing module. The editing module parses a medical record stored in the memory, determines a first outcome for coding the medical record based on one or more pre-defined rules, determines a second outcome for coding the medical record based on one or more adaptive rules, wherein the adaptive rules are defined based on statistical machine learning based on processing of other medical records, selects between the first and second outcomes, and causes output on an output device based on the selected outcome.

In another example, this disclosure describes a device for processing medical data, the device comprising means for parsing a medical record via the one or more computers, means for determining a first outcome for coding the medical record based on one or more pre-defined rules, means for determining a second outcome for coding the medical record based on one or more adaptive rules, wherein the adaptive rules are defined based on statistical machine learning based on processing of other medical records, means for selecting between the first and second outcomes, and means for causing output for coding the medical record based on the selected outcome.

In another example, this disclosure describes a computer-readable storage medium comprising instructions that when executed in a processor cause the processor to process medical data, wherein upon execution the instructions cause the processor to parse a medical record via the one or more computers, determine a first outcome for coding the medical record based on one or more pre-defined rules, determine a second outcome for coding the medical record based on one or more adaptive rules, wherein the adaptive rules are defined based on statistical machine learning based on processing of other medical records, select between the first and second outcomes, and cause output for coding the medical record based on the selected outcome.

The details of one or more examples of this disclosure are set forth in the accompanying drawings and the description below. Other features, objects, and advantages associated with the examples will be apparent from the description and drawings, and from the claims.

### BRIEF DESCRIPTION OF DRAWINGS

FIG. 1 is a block diagram illustrating an example of a stand-alone computer system for coding medical data consistent with this disclosure.

FIG. 2 is a block diagram illustrating an example of a distributed system for coding medical data consistent with this disclosure.

FIGS. 3-8 are exemplary computer screen shots that may illustrate one or more features of this disclosure.

FIG. 9 is an exemplary depiction of a physician's documentation request that may be generated according to one or more techniques of this disclosure.

FIG. 10 is a flow diagram illustrating a technique of this disclosure.

FIG. 11 is a table illustrating medical codes, suppression codes, and key word terms that may be associated with certain codes.

FIGS. 12 and 13 are additional flow diagrams illustrating techniques of this disclosure.

### DETAILED DESCRIPTION

This disclosure describes systems and techniques for processing medical data via one or more computers. The systems and techniques may be used by a medical reviewer (sometimes referred to as a “documentation specialist”). The documentation specialist may be assigned the task of reviewing medical records for purposes of billing a payer and ensuring accuracy in the medical records. The payer may be a governmental agency, such a Medicare or Medicaid, or a private entity, such as an insurance company. The documentation specialist may be a nurse, clinician, administrative person, or any person given the task of reviewing medical records, and verifying or updating medical codes in the medical documentation. In general, the documentation specialist typically reviews medical records and ensures that the medical records include the correct medical codes associated with the medical tasks or medical conditions defined in the medical records.

Unfortunately, medical records can be long, complicated, and sometimes incomplete. This can make the coding and review process very time consuming and difficult for a documentation specialist. The techniques and systems described herein can help to automate (or partially automate) the coding process associated with medical record review. In this manner, the process of coding medical records and verifying codes in medical records can be improved and/or simplified. The techniques may apply one or more rules to define when documentation for medical records is sufficient and when further review of the medical records is needed. The rules may define when the documentation specialist should review the medical records and may automate the process by avoiding the display of medical records to the documentation specialist when the documentation in the medical records is sufficient. The rules may further define when physician review is needed, and may automate the process by avoiding physician review when the documentation in the medical records is sufficient or when review by the documentation specialist may suffice prior to (and possibly in lieu of) physician review. Physician review of medical records is generally undesirable when it can be avoided as it is time consuming, labor intensive, and adds cost. Additional techniques are also described, which may rely on machine learning to replace one or more of the rules described herein. With machine learning, the rules or techniques may adapt over time based on statistics associated with the selections or activities of documentation specialists with respect to coding of prior medical records.

As described in greater detail below, the methods of this disclosure may be performed by one or more computers. The methods may be performed by a stand-alone computer, or may be executed in a client-server environment in which a documentation specialist views medical records at a client computer. In the later case, the client computer may communicate with a server computer. The server computer may store the medical records and apply the techniques of this disclosure to facilitate medical record review and coding addition or modification by the documentation specialist at the client computer.

In one example, a method may include identifying, via one or more computers, a medical code within a medical record, and identifying, via the one or more computers, whether the medical code is one of a plurality of specified medical codes or one of a plurality of unspecified medical codes. The specified medical codes may be defined as sufficient to reflect and accurately represent a medical condition to a payer and the unspecified medical codes are defined as requiring additional information for medical condition clarity. If the medical code is one of the specified medical codes, the one or more computers may avoid display of clinical edit options for the medical record without generating a query for further input by a physician. If the medical code is one of the unspecified medical codes, the one or more computers may determine whether one of a plurality of suppression codes associated with the medical code appears in the medical record. If one of the suppression codes appears in the medical record, the one or more computers may avoid display of the clinical edit options for the medical record without generating the query for further input by the physician. If one of the suppression codes does not appear in the medical record, the method may include searching for one or more key terms in the medical record via the one or more computers. If one or more key terms are present in the medical record, the method may include causing display of the clinical edit options for the medical record via the one or more computers. If one or more key terms are present in the medical record, the method may include generating the query for further input by the physician via the one or more computers.

It should be appreciated that implementations of the disclosed subject matter offer numerous advantages over existing systems. For instance, as mentioned elsewhere existing systems process large amounts of data and by preventing the processing of queries based on the presence of a suppression code as described, the underlying computing technology is improved. For example, the use of suppression codes can reduce the computational overhead for systems configured as described because generating queries that are not needed is avoided. As another example, and as described in more detail below, the graphical user interface of the system is improved because information is automatically presented in a particular way improving aspects of how the data is visualized and used, improving both the accuracy and usability of the system in the process. For instance, because suppressed information is not presented, only information that is necessary to a particular task is presented in the graphical user interface.

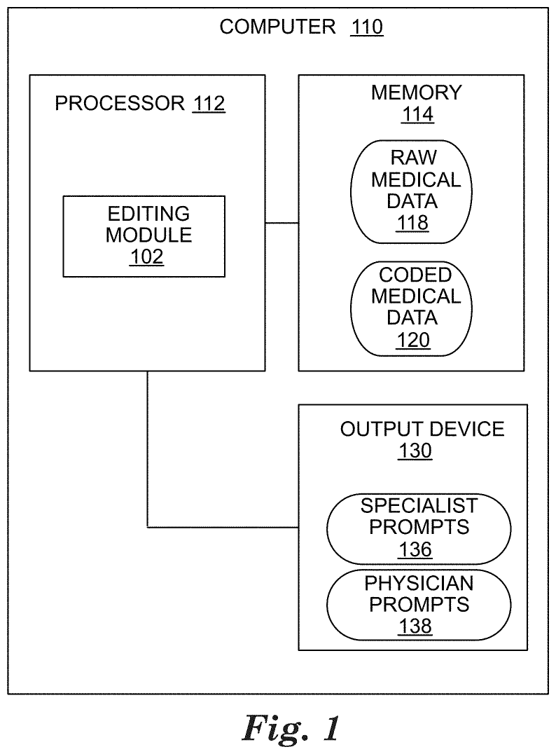

FIG. 1 is a block diagram of illustrating an example of a stand-alone computerized system for coding medical data consistent with this disclosure. The system comprises computer 110 that includes a processor 112, a memory 114, and an output device 130. Computer 110 may also include many other components. The illustrated components are shown merely to explain various aspects of this disclosure.

The output device 130 may comprise a display screen, although this disclosure is not necessarily limited in this respect, and other types of output devices may also be used. Memory 114 stores raw medical data 118 comprising medical records. Processor 112 is configured to include an editing module 102 that executes techniques of this disclosure with respect to raw medical data 118, and in some cases, editing module 102 may generate coded medical data 120 comprising edited medical records.

Processor 112 may comprise a general-purpose microprocessor, a specially designed processor, an application specific integrated circuit, a field programmable gate array, a collection of discrete logic, or any type of processing device capable of executing the techniques described herein. In one example, memory 114 may store program instructions (e.g., software instructions) that are executed by processor 112 to carry out the techniques described herein. In other examples, the techniques may be executed by specifically programmed circuitry of processor 112. In these or other ways, processor 112 may be configured to execute the techniques described herein.

Output device 130 may comprise a display screen, and may also include other types of output capabilities. In some cases, output device 130 may generally represent both a display screen and a printer in some cases. Editing module 102 may be configured to cause output device 130 to output specialist prompts 136 and physician prompts 138. Specialist prompts 136 may be generated, e.g., as output on a display screen, so as to allow the documentation specialist to add or modify coding edits to the medical records. In this manner, coded medical data 120 may be generated and stored based on raw medical data 118 and based on additional information from a documentation specialist operating in response to specialist prompts 136 generated at output device 130. In addition, physician prompts 136 may be generated, e.g., as output on a display screen or printouts of one or more request forms for a physician. This can allow a physician to provide additional information so as to improve and/or supplement the medical records, when necessary. The techniques of this disclosure may serve to automate the coding and the review process with respect to medical records, minimizing both specialist prompts 136 and physician prompts 138. For example, specialist prompts 136 can be minimized to situations in which the medical records do not include the necessary information, but do include key words from which a documentation specialist may be able to add or modify medical codes based on the information in the record. Physician prompts 138 may be minimized to situations in which the medical records do not include the necessary information, and also lack sufficient key words from which a documentation specialist would be able to add or modify medical codes based on the information in the record.

In one example, editing module 102 identifies a medical code within a medical record stored in the memory 114. The medical record may be one of many medical records within raw medical data 118 needing review by a documentation specialist. Editing module 102 identifies whether the medical code is one of a plurality of specified medical codes or one of a plurality of unspecified medical codes. The specified medical codes are defined as sufficient to reflect and accurately represent a medical condition to a payer and the unspecified medical codes are defined as requiring additional information for medical condition clarity. The payer typically comprises either a governmental payer, or an insurance company, although the techniques of this disclosure may apply to other payers.

If the medical code is one of the specified medical codes, editing module 102 avoids causing display of clinical edit options via specialist prompts 136 on output device 130 for the medical record. In this case, editing module 102 also avoids the generation of a query for further input by a physician, e.g., avoids generating physician prompts 138 on output device 130.

If the medical code is one of the unspecified medical codes, editing module 102 determines whether one of a plurality of suppression codes associated with the medical code appears in the medical record stored in raw medical data 118 in memory 114. If one of the suppression codes appears in the medical record editing module 102 avoids causing display of clinical edit options via specialist prompts 136 on output device 130 for the medical record. In this case, editing module 102 also avoids the generation of a query for further input by a physician, e.g., avoids generating physician prompts 138 on output device 130.

At this point, if one of the suppression codes does not appear in the medical record, editing module 102 searches for one or more key terms in the medical record. If one or more key terms exist in the medical record, editing module 102 generates specialist prompts 136 on output device, e.g., causing display of the clinical edit options for the medical record stored in raw medical data 118 of memory 114. When code additions or modifications are received from a documentation specialist, editing module 102 stores an edited version of the medical record in coded medical data 120 within memory 114. On the other hand, if one or more key terms are present in the medical record and do not contain sufficient detail as to define a code, editing module 102 generates physician prompts 138 on output device, e.g., causing display or printout of a query for further input by the physician.

The medical codes within the medical records may comprise codes defined by the International Classification of Diseases (ICD), such as ICD-9 codes or ICD-10 codes, although the techniques are not necessarily limited to ICD medical codes and could apply with respect to other types of medical codes as would be apparent to one of skill in the art. In particular, other medical codes may be used with the techniques of this disclosure, particularly for billing to insurance companies or other non-governmental organizations, which may define their own code system or may adopt that of the ICD. Like the medical codes, the suppression code may also be defined by the ICD, wherein the suppression codes are more specific than the medical codes. According, a given suppression code may override and “suppress” a broader medical code by providing more specific information on a given condition or procedure coded in the medical record.

In some examples, the key terms are pre-defined, and editing module 102 automatically searches for the key terms within the medical record when one of the suppression codes does not appear in the medical record. In other examples, at least some of the associations between key terms and queries may be adaptively defined, in which case machine learning techniques may be used over time to associate key terms with queries to medical records that are made by the documentation specialist. Accordingly, in this case editing module 102 may adaptively define at least some of the key terms based on previous searches for terms performed by one or more users (e.g., other documentation specialists that performed review and edits or similar types of medical records). For example, editing module 102 may cause the display of possible terms to the one or more users (e.g., as specialist prompts 136), and editing module 102 may then search for ones of the possible terms within a medical record based on selections by the one or more users (e.g., user input in response to specialist prompts 136). In this case, one or more of the associations between key terms and queries may be adaptively defined by editing module 102 over time based on the selections of the possible terms by the one or more users. Moreover, once one or more of the associations between key terms and queries are adaptively defined over time based on the selections of the possible terms by the one or more users, editing module 102 may be configured to automatically search for the adaptively defined associations between key terms and queries when one of the suppression codes does not appear in the medical record. In this manner, machine learning techniques may be used over time to associate key terms with selections and/or queries to medical records made by documentation specialists. Additional machine learning techniques are also discussed below.

When causing display of the clinical edit options for the medical record, editing module 102 may cause any of a wide variety of specialist prompts 136 to appear on output device 130. In some examples, specialist prompts 136 may display of at least a portion of data from the medical record in raw medical data 118 to allow for review by a documentation specialist. Once code edits e.g., additions and/or modifications are reviewed and confirmed by the documentation specialist, editing module 102 may cause the edited version of the medical record to be stored in memory 114 as coded medical data 120.

When generating a query for further input by the physician, editing module 102 may automatically or manually, through a documentation specialist, generate physician prompts 138. Physician prompts 138 may comprise a physician documentation request that requests additional details for the medical record. As examples, the requested details may pertain to the medical code, the suppression code, or one or more key terms. In this way, physician prompts 138 can be automated, yet limited to situations in which physician input is actually needed. Accordingly, unwanted or unnecessary queries to the physician can be substantially minimized.

The system of FIG. 1 is a stand-alone system in which processor 112 that executed editing module 102 and output device 130 that outputs specialist prompts 136 and physician prompts 138 reside on the same computer 110. However, the techniques of this disclosure may also be performed in a distributed system that includes a server computer and a client computer. In this case, the client computer may communicate with the server computer via a network. The editing module may reside on the server computer, but the output device may reside on the client computer. In this case, when the editing module causes display prompts, the editing module causes the output device of the client computer to display the prompts, e.g., via commands or instructions communicated from the server computer to the client computer. The editing module may simply avoid such commands or instructions if display of the prompts at the output device is avoided.

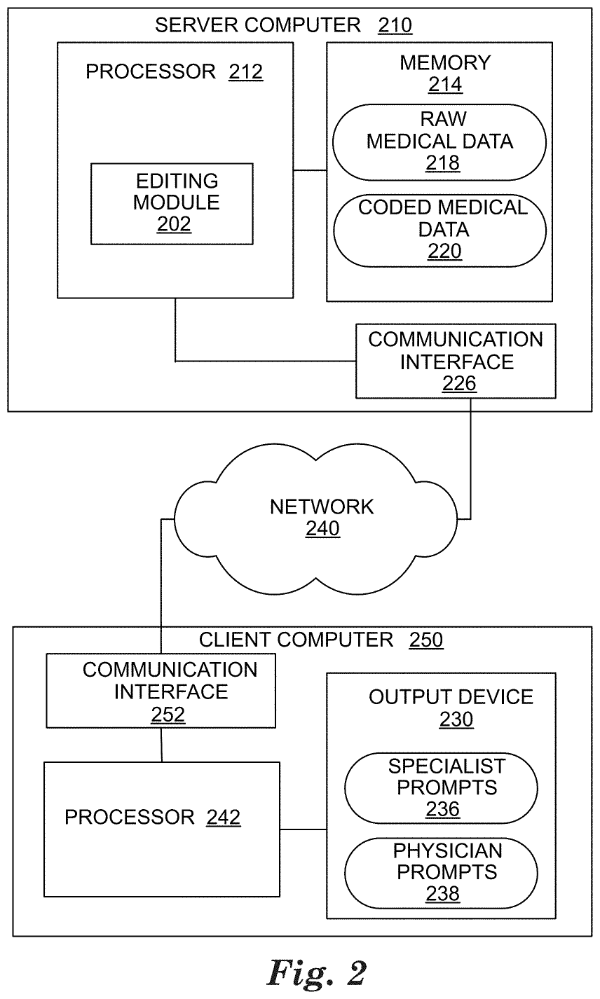

FIG. 2 is a block diagram of a distributed system that includes a server computer 210 and a client computer 250 that communicate via a network 240. In the example of FIG. 2, network 240 may comprise a proprietary on non-proprietary network for packet-based communication. In one example, network 240 comprises the Internet, in which case communication interfaces 226 and 252 may comprise interfaces for communicating data according to transmission control protocol/internet protocol (TCP/IP), user datagram protocol (UDP), or the like. More generally, however, network 240 may comprise any type of communication network, and may support wired communication, wireless communication, fiber optic communication, satellite communication, or any type of techniques for transferring data between a source (e.g., server computer 210) and a destination (e.g., client computer 240).

Server computer 210 may perform the techniques of this disclosure, but the documentation specialist (i.e., the user) may interact with the system via client computer 250. Server computer 210 may include a processor 212, a memory 214, and a communication interface 226. Client computer 250 may include a communication interface 252, a processor 242 and an output device 230. Of course, client computer 250 and server computer 210 may include many other components. The illustrated components are shown merely to explain various aspects of this disclosure.

Output device 230 may comprise a display screen, although this disclosure is not necessarily limited in this respect and other output devices may also be used. Memory 214 stores raw medical data 218 comprising medical records. Processor 212 of server computer 210 is configured to include an editing module 202 that executes techniques of this disclosure with respect to raw medical data 218, and in some cases, editing module 202 may generate coded medical data 220 comprising edited medical records.

Processors 212 and 242 may each comprise a general-purpose microprocessor, a specially designed processor, an application specific integrated circuit, a field programmable gate array, a collection of discrete logic, or any type of processing device capable of executing the techniques described herein. In one example, memory 214 may store program instructions (e.g., software instructions) that are executed by processor 212 to carry out the techniques described herein. In other examples, the techniques may be executed by specifically programmed circuitry of processor 212. In these or other ways, processor 212 may be configured to execute the techniques described herein.

Output device 230 on client computer 250 may comprise a display screen, and may also include other types of output capabilities. For example, output device 230 may generally represent both a display screen and a printer in some cases. Editing module 202 may be configured to cause output device 230 of client computer 250 to output specialist prompts 236 and physician prompts 238. Specialist prompts 236 may be generated, e.g., as output on a display screen, so as to allow the documentation specialist to add or modify codes based on the medical records or provide additional information from the medical record to help clarify the query. In this manner, coded medical data 220 may be generated and stored based on raw medical data 218 and based on additional information, e.g., code edits, additions or modifications, from a documentation specialist operating in response to specialist prompts 236 generated at output device 230 of client computer 250. In addition, physician prompts 236 may be generated, e.g., as output on a display screen or printouts of one or more request forms for a physician. This can allow a physician to provide additional information so as to improve and/or supplement the medical records, when necessary. Again, the techniques of this disclosure may serve to automate the coding and the review process with respect to medical records, minimizing both specialist prompts 236 and physician prompts 238. For example, specialist prompts 236 can be minimized to situations in which the medical records do not include the necessary information, but do include key words from which a documentation specialist may be able to review based on the information in the record. Physician prompts 238 may be minimized to situations in which the medical records do not include the necessary information, and also lack sufficient key words from which a documentation specialist would be able to review based on the information in the record.

Similar to the stand-alone example of FIG. 1, in the distributed example of FIG. 2, editing module 202 identifies a medical code within a medical record stored in the memory 214. The medical record may be one of many medical records within raw medical data 218 needing review by a documentation specialist at client computer 250. Editing module 202 identifies whether the medical code is one of a plurality of specified medical codes or one of a plurality of unspecified medical codes. The specified medical codes are defined as sufficient to reflect and accurately represent a medical condition to a payer and the unspecified medical codes are defined as requiring additional information for medical condition clarity. Again, the payer typically comprises either a governmental payer, or an insurance company, although the techniques of this disclosure may apply to other payers.

If the medical code is one of the specified medical codes, editing module 202 avoids causing display of clinical edit options via specialist prompts 236 on output device 230 of client computer 250 for the medical record. In this case, editing module 202 also avoids the generation of a query for further input by a physician, e.g., avoids generating physician prompts 238 on output device 230 of client computer 250. Again, communication interfaces 226 and 252 allow for communication between server computer 210 and client computer 250 via network 240. In this way, editing module may execute on server computer 210 but the output may appear on output device 230 of client computer. A documentation specialist operating on client computer 250 may log-on or otherwise access editing module of server computer 210, such as via a web-interface operating on the Internet or a propriety network, or via a direct or dial-up connection between client computer 250 and server computer 210. In some cases, data displayed on output device 230 (including any specialist prompts 238 or physician prompts 238) may be arranged in web pages served from server computer 210 to client computer 250 via hypertext transfer protocol (HTTP), extended markup language (XML), or the like.

If the medical code is one of the unspecified medical codes, editing module 202 determines whether one of a plurality of suppression codes associated with the medical code appears in the medical record stored in raw medical data 218 in memory 214. If one of the suppression codes appears in the medical record editing module 202 avoids causing display of clinical edit options via specialist prompts 236 on output device 230 for the medical record. In this case, editing module 202 also avoids the generation of a query for further input by a physician, e.g., avoids generating physician prompts 238 on output device 230 of client computer 250.

At this point, if one of the suppression codes does not appear in the medical record, editing module 202 searches for one or more key terms in the medical record. If one or more key terms exist in the medical record, editing module 202 generates specialist prompts 236 on output device 230 of client computer 250, e.g., causing display of the clinical edit options for the medical record stored in raw medical data 218 of memory 214. When code additions or modifications are received from a documentation specialist operating on client computer 250, editing module 202 stores an edited version of the medical record in coded medical data 220 within memory 214. On the other hand, if one or more key terms are present in the medical record and do not contain sufficient detail as to define a code, editing module generates physician prompts 238 on output device, e.g., causing display or printout of a query for further input by the physician. Both the presence of key terms and the absence of key terms in the medical record may be used, in some cases, to determine the outcome (e.g., display of edit options to the documentation specialist or display or output of a query for further input by the physician.

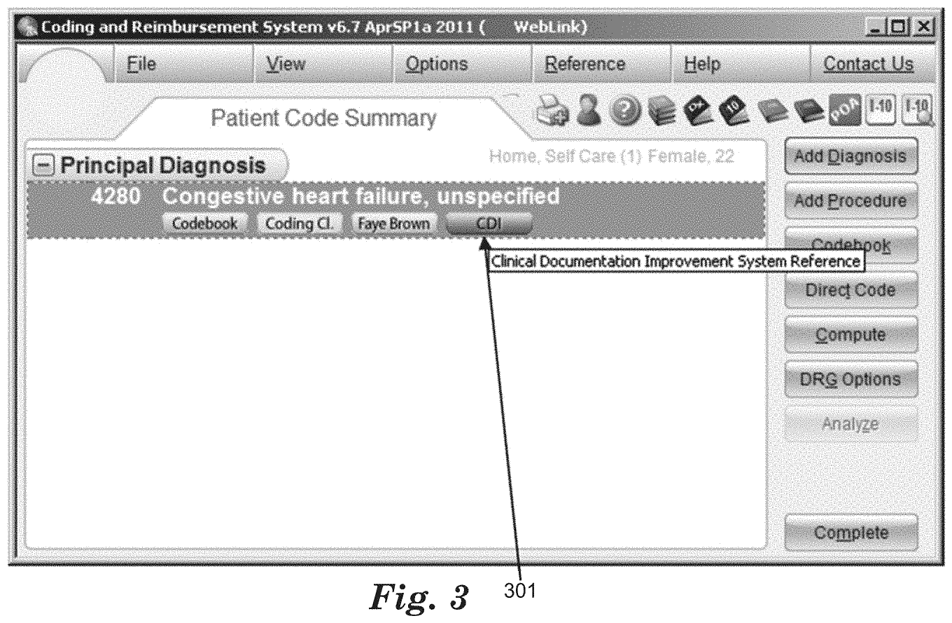

FIGS. 3-8 are exemplary computer screen shots that may illustrate one or more features of this disclosure. These screen shots may be delivered to output device 130 of computer 110 shown in FIG. 1 or output device 230 of client computer 250 shown in FIG. 2. In each case, the screen shots may be generated as part of an editing routine (e.g., editing module 102 or 202) executed by processor 112 of computer 110 shown in FIG. 1, or executed processor 212 of client computer 250 shown in FIG. 2.

It should be appreciated that the examples illustrated in FIGS. 3-8 provide numerous advantages over existing systems. As discussed elsewhere, one of the problems associated with coding medical data is that it is a difficult and time-consuming process that is error-prone. Part of the reason for this is the volume of information that a human user must review or otherwise access in order to properly perform a manual review of the information. The example graphical user interfaces illustrated and described overcome this problem by both limiting the information presented and also by presenting the information in a particular way that allows for not only an efficient review of the relevant information, but also improves the overall accuracy of the system because the information is structured and presented in such a way to limit the opportunities for user error.

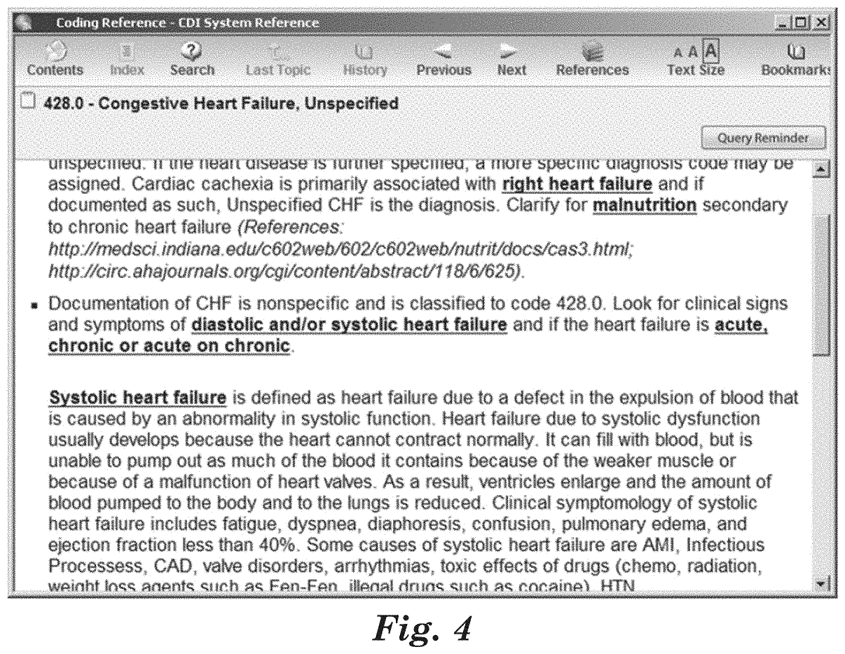

A documentation specialist may select a patient record in order to review the documentation that physician created in response to a patient visit. The documentation specialist may also be referred to as a coder, a reviewer, or a user of the system described herein. After the documentation specialist reads the patient record, they may add the diagnosis code, such as 428.0 to code unspecified congestive heart failure (CHF). As shown in FIG. 3, this may result in automatic generation of a clinical document improvement button 301. Button 301 is only present if the If the documentation specialist clicks on clinical document improvement button 301, this may result in the display of a clinical document improvement (CDI) reference material for code 428.0. An exemplary screen shot of a CDI reference material for cognitive heart failure under coded 428.0 is illustrated in FIG. 4.

Manual review of CDI reference material can be difficult and time-consuming for a documentation specialist. Review of CDI reference material like that shown in FIG. 4 may inform the documentation specialist of key words such as dyspnea, pulmonary edema, ejection fraction <40%, and so forth. In manually reviewing medical records based on this CDI reference material, the documentation specialist may search for clinical findings that match the key words, or may identify the need for the physician to provide and/or clarify the documentation. Upon finding documentation improvement opportunities, the documentation specialist may manually generate a query for the physician. This can result in a time-intensive process that requires careful and thoughtful examination of the documentation in order to catch all documentation improvement opportunities. The techniques of this disclosure may automate some or all of this documentation coding process. Indeed, an automated (or at least partially automated) clinical documentation system can provide the documentation specialist with significant productivity enhancements, especially when delivering auto-suggestion capabilities (e.g., suggestions via specialist prompts or physician prompts) consistent with the rule-based automations described herein. As the documentation specialist adds diagnosis or procedure codes, the system may automatically search through the documentation looking for CDI clinical edits that the system can suggest to the documentation specialist. In some examples, the system may notify the documentation specialist of CDI opportunities by displaying a particular icon on a code summary screen.

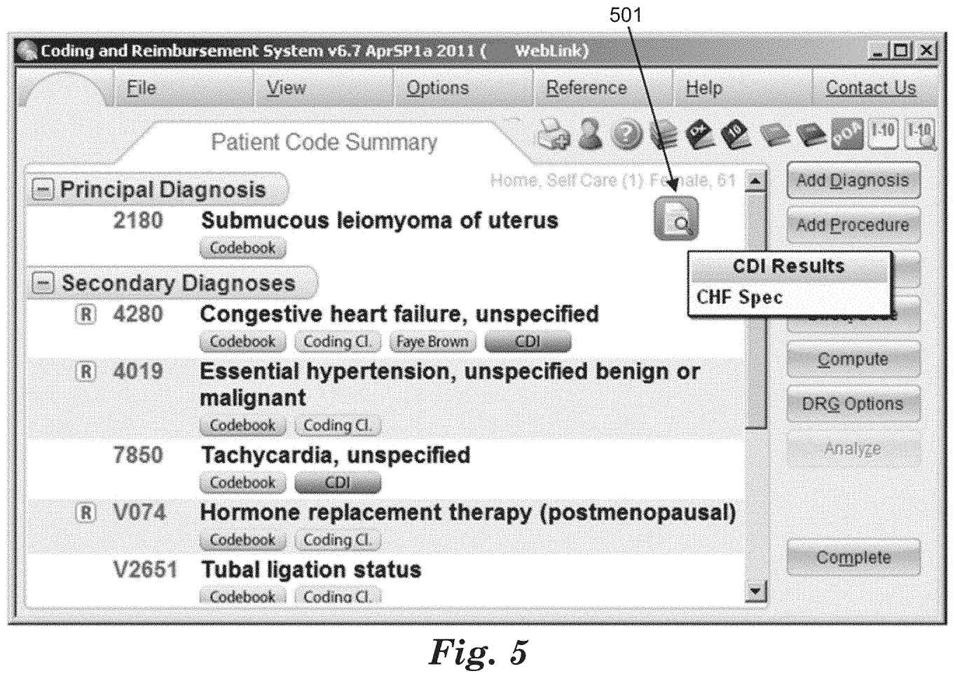

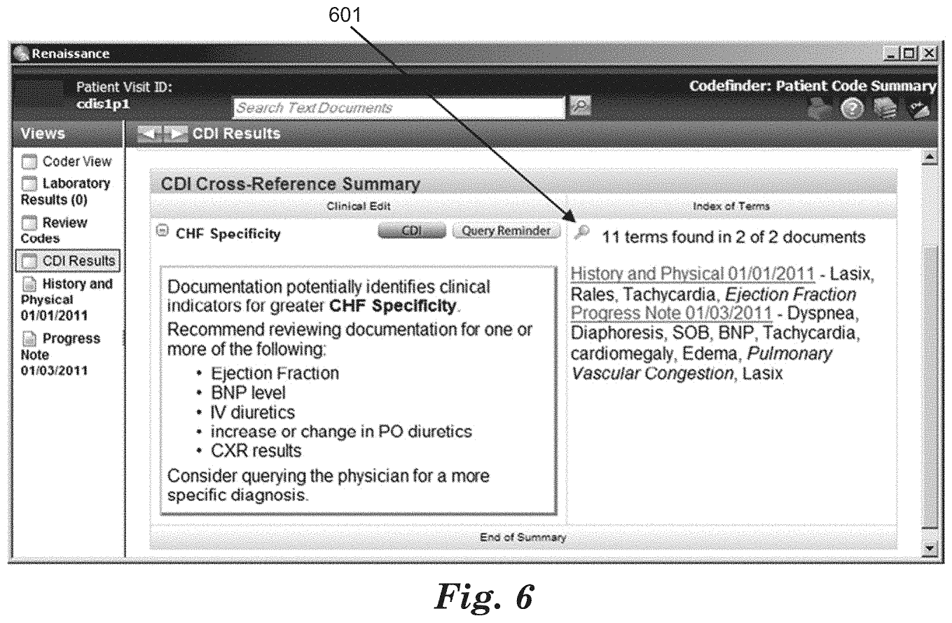

For example, FIG. 5 illustrates a screen shot in which a CDI icon 501 is displayed to the documentation specialist in conjunction with a particular diagnosis code or description (in this case, code 428.0 for congestive heart failure). The documentation specialist may click on CDI icon 501 shown in FIG. 5 as part of a graphical user interface (GUI), in order to display one or more CDI clinical edit opportunities, such as shown in the screen shot of FIG. 6.

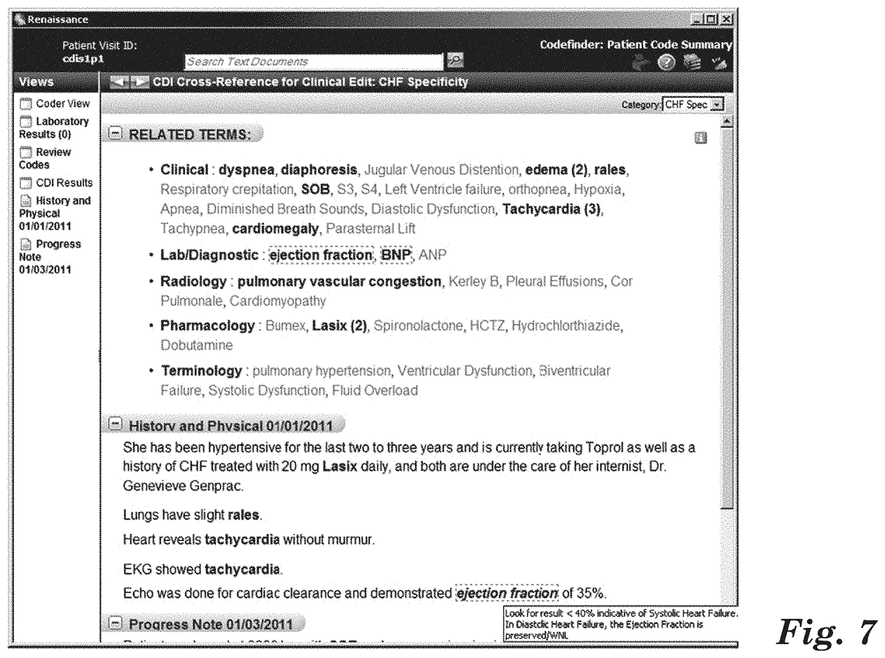

For example, the screen shot of FIG. 6 illustrates an exemplary presentation of CDI clinical edit opportunities with terms highlighted for further review. On the right side of the screen shot of FIG. 6, terms that were found in the documentation can be displayed. By clicking on the magnifying glass icon 601, the documentation specialist can be provided a cross-reference view of that clinical edit, as shown in the screen shot of FIG. 7.

In the cross-reference view depicted in FIG. 7, the system may display all of the related terms that were searched with respect to a medical record. In addition, terms that were found in the medical record may be bolded, as shown in FIG. 7. Terms that have a dotted-line surrounding them may include additional information available, such as by so-called “flyover” where the cursor is passed over the terms to cause display of the additional information. This flyover is shown as the display of information indicating that ejection fraction <40% is indicative of Systolic Heart Failure. In this way, an improved graphical user interface is realized over existing systems because only those elements that warrant user review are indicated therein. Stated differently, while existing systems provide more information than is necessary—and therefore must rely on the user to determine what is important and what can be ignored—systems configured as described provide a more curated user interface that improves the visualization of the data and improves the system's useability. Ultimately, because the user is not responsible for filtering extraneous information from the GUI, the GUI improves the accuracy of the system by allowing the user to focus on what is needed for a particular coding task.

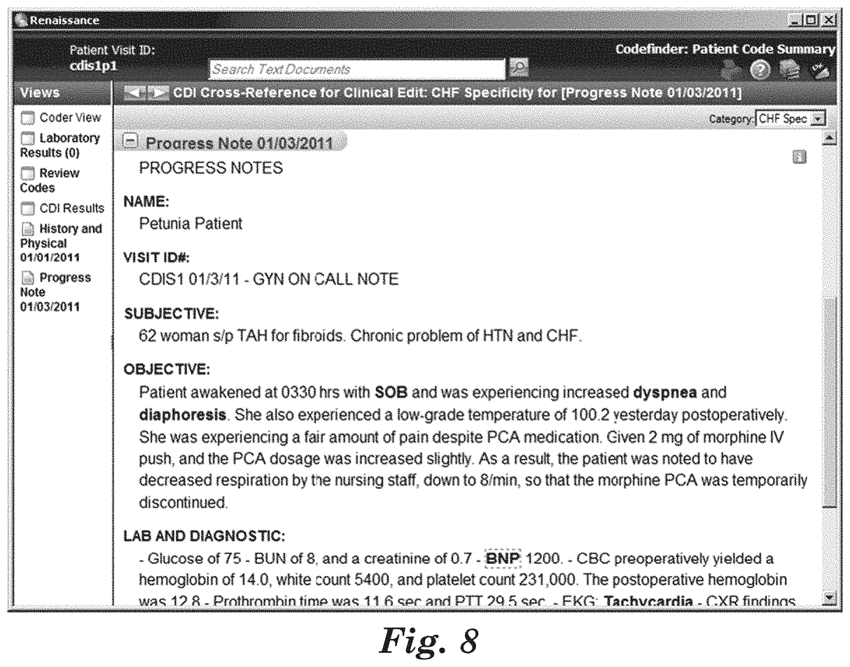

The documentation specialist may also be able to review the entire medical record by clicking on the document title, e.g., a GUI link labeled as “progress notes 1/03/2011” on the left hand side of FIG. 7. This may result in display of the entire medical record for review by the documentation specialist as shown in FIG. 8. The record may be displayed with any CDI terms found in the document being shown in bold.

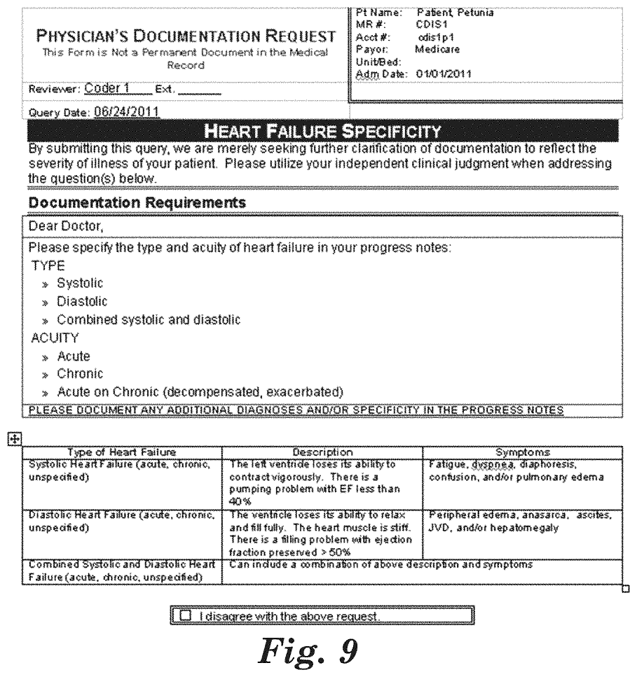

FIG. 9 is an example physician's documentation request, which may be generated as a physician prompt consistent with this disclosure. This physician's documentation request may be automatically generated or manually generated by the documentation specialist, as described herein, when necessary, but may be avoided when possible according to the rules described herein. The physician's documentation request shown in FIG. 9 may be output electronically and delivered to the physician electronically, or may be output as a manual printout in some examples. This is simply one example of a query that can be created and sent to the physician for additional input with respect to a given medical record.

It should be appreciated that by avoiding the need to initiate the request according to the rules described herein, computing resources are conserved because the computing system implementing these techniques does not perform steps that have been automatically determined to be unnecessary. This too provides technological advantages not seen in existing systems because, e.g., the automatic determinations as described yield a faster processing time over existing systems while improving, not sacrificing, the accuracy as a result.

The example of FIG. 9 includes input from the physician, identifying that the patient has acute systolic heart failure. Based on this additional information from the physician, the documentation specialist can again review the patient record, and recode 428.0 to a new code (428.21) to identify acute systolic heart failure. In this case, the CHF specificity clinical edit may disappear from the clinical edit options because the documentation specialist has added a new code with greater specificity. In other cases, medical codes could be replaced and removed from medical documentation instead of being recoded. As an example, if it was discovered that a patient was diagnosed with stage 3 chronic kidney disease 585.3 instead of chronic renal insufficiency 585.9.

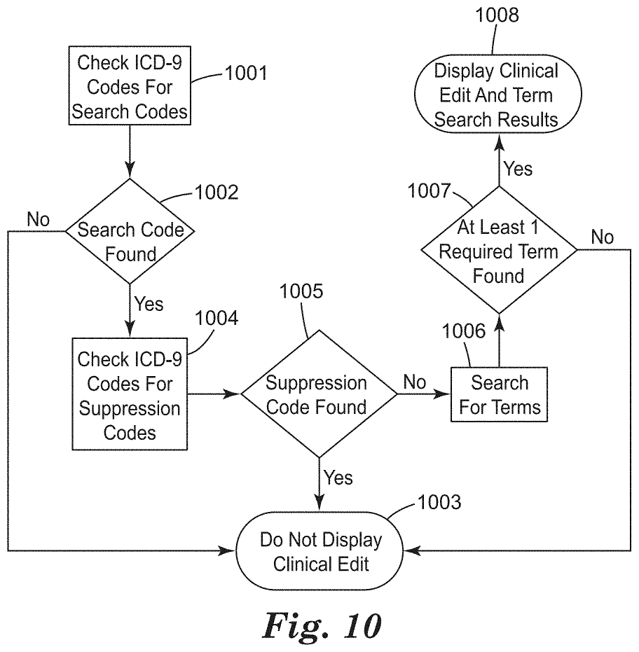

FIG. 10 is an exemplary flow diagram illustrating a technique consistent with this disclosure. FIG. 10 will be described from the perspective of computer 110 of FIG. 1, although the system of FIG. 2 or other systems could also be used to perform such techniques. As shown in FIG. 10, editing module checks ICD-9 codes for “search codes” (1001). The lack of any search code (“no” 1002) may identify a respective ICD-9 code as being specified, wherein specified medical codes are defined as sufficient to reflect and accurately represent a medical condition to a payer. In this case, editing module 102 does not cause the display of any clinical edit options to the documentation specialist (1003), e.g., since sufficient information should be included for the medical record in the form of a specified ICD-9 code.

If a search code is found (“yes” 1002), editing module 102 may check the ICD-9 codes for suppression codes (1004). If a suppression code is found (“yes” 1005), then editing module 102 does not cause the display of any clinical edit options to the documentation specialist (1003), e.g., since sufficient information should be included for the medical record in the form of a suppression code. However, if a suppression code is not found (“no” 1005), then editing module 102 may search for key terms in the medical record (1006). At this point, if at least one required term is found in the medical record (“yes” 1007), then editing module 102 does not cause the display of any clinical edits to the documentation specialist (1003), e.g., since sufficient information should be included for the medical record in the form of a required term (possibly in conjunction with other elements in the medical record). However, if at this point any required terms are present in the medical record (“no” 1007) and do not contain sufficient detail as to define a code, then editing module 102 may cause output device 130 to display the clinical edits and search terms (1008), e.g., which may correspond to the display of specialist prompts 136. It will be apparent to one of skill in the art that other medical classification systems may be used in place of ICD-9 codes.

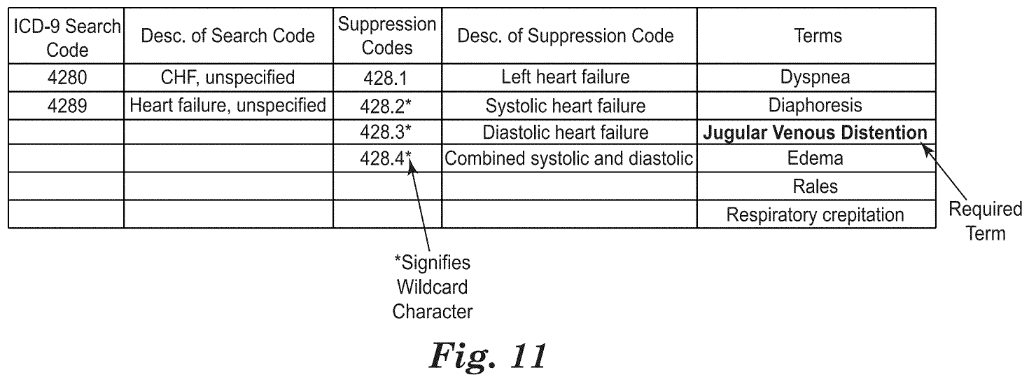

FIG. 11 is a table showing some exemplary ICD-9 search codes, and associated descriptions. FIG. 11 also shows some exemplary suppression codes, some of which may include a wildcard character that is designated as an asterisk e.g., 428.3*. The wildcard character implies a multitude of codes where any within the range could be a suppression code. In addition, FIG. 11 also shows corresponding descriptions for the suppression codes. Finally, FIG. 11 also shows exemplary key terms. The bolded terms “jugular venous distension” may define required terms (or a required set of terms that collectively define a required phrase) that may be clinical indicators of diastolic heart failure (suppression 428.3*). Accordingly, in the process of FIG. 10, if codes are lacking from the medical record, the presence of the key terms “jugular venous distension” results in the display of clinical edit options to the documentation specialist. The documentation specialist, based upon displayed clinical edit options, determines whether or not a clinical edit warrants a physician query. In essence, clinical edit options are displayed to provide clarity and enable documentation specialists to generate queries when documentation is insufficient to reflect and accurately represent a medical condition. For some queries, the determination by the documentation may be automated through statistical machine learning techniques where the absence of suppression codes and presence of key terms from the medical record have a high probability of generating a particular query.

According to the techniques described herein, instead of having a time intensive manual process for entering codes, reviewing the CDI reference information and searching the documentation for CDI opportunities the documentation specialist may reap benefits of an automated process. With the CDI enhancement and auto-suggestions of codes, the documentation specialists may begin coding sessions with codes and CDI clinical edits already present for their review and consideration, which can result in significant productivity improvement for the documentation specialists.

In some example, the steps taken to generate either suppression or non-suppression codes that ultimately lead to a query suggestion can be reduced through either auto-suggesting the codes, or bypassing one or more choices in a decision-tree logic based encoder implemented e.g., as part of the described editing module. By annotating clinical terminology in the patient documentation and integrating decision-tree choices (referred to herein as coding paths) directly with the annotations, productivity improvements can be achieved. Essentially, the coding path choices can be prompted on an exception basis. Bypassed steps may include the manual entry of clinical terms and the manual selection of coding path prompts that can be satisfied based on the clinical terminology annotated in the medical record.

In a manual process, the user may perform the following steps:

Step 1: Manually enter a clinical term to begin a coding path.

Step 2: Manually select a choice for each prompt in the decision tree (multiple prompts are the norm).

Step 3: Manually obtain a code.

Step 4: Manually validate the code based on documentation and other codes present.

In one automated example, annotations may be embedded in coding paths. With the coding paths embedded within a document's annotated clinical terminology, the manual steps above may be changed as follows:

Step 1: The clinical term can be automatically applied to begin a coding path based on selection of an annotation, essentially eliminating Step 1 from above.

Step 2: One or more prompts in the coding path can be auto-selected based on the annotation selection. This can reduce the manually selections needed.

Step 3: The code is obtained.

Step 4: The code can be validated based on documentation present. The validation can be expedited by document view and searching capabilities for relevant clinical terminology.

In another automated example, auto-suggested codes can be generated. In this case, with auto-suggested codes, steps 1 and 2 above can be eliminated from the perspective of the documentation specialist. The system may identify a code along with evidence for the clinical specialist to validate the code. The steps above may be revised as follows:

Step 1: Eliminated.

Step 2: Eliminated.

Step 3: A code is automatically suggested to the documentation specialist based on data in the medical record.

Step 4: The code is validated by the documentation specialist based on summary evidence generated for each suggested code. The validation is expedited by document view and searching capabilities for relevant clinical terminology. The validation of the code is also expedited by auto-generating a coding path associated for that code. This coding path can be presented in a single “at-a-glance” summary view that also allows the coder to quickly navigate within the path to a more appropriate code, if needed.

In additional examples, one or more of the techniques and rules described herein may be replaced or supplemented with machine learning techniques based on statistics. In this case, the actions of documentation specialists can be saved and the computer may learn and adapt future coding suggestions based on statistics associated with the prior actions of documentation specialists.

In both a rules-based approach or a statistical machine learning approach, it may be desirable to determine one of three outcomes: (1) automatically coding the document (and either sending the document directly to billing or to a human review for final approval), (2) issuing one or more specificity queries back to the physician to improve the documentation, or (3) determining that the automated system was not confident in its prediction and sending the documentation to a human reviewer to choose outcome (1) or (2). In some systems, if the documentation is complete enough that a human coder (i.e., a documentation specialist) would not issue a specificity query to a physician, outcome (1) may be chosen, and only when the documentation is missing key information may outcome (2) be chosen, since it is often considered costly and undesirable to query the physician. Therefore, systems that reduce the number of situations with outcome (3), as well as systems reducing the number of documents mistakenly given outcome (2) when they should have been given outcome (1) or visa-versa, may improve conventional coding systems.

In one exemplary machine learning approach, a computer may gather data on the actions of the documentation specialist, and observe or analyze the patient documentation and the outcome (e.g., outcomes (1) or (2) mentioned above). The computer may also observe or analyze any codes or queries generated in each case. The computer may also process documents to identify linguistic and clinical evidence, including one or more of clinical terminology, non-clinical terminology, negation, ambiguity, semantic relationships of identified terms, sentence structure, word order, temporal references, document sections and document structure. The computer may then train one or more machine learning models based on the gathered data to determine statistical relationships between the linguistic and clinical evidence and any resulting action (e.g., outcome (1) or (2), codes generated, or specificity queries generated). By performing such tasks, the choice between outcomes (1), (2) or (3) can be made. In some cases, the computer may implement a support vector machine in order to implement one or more of these machine learning techniques.

For new patient documentation, a statistical machine learning approach to coding may apply statistical models described above to predict the outcome, one or more codes and any queries that may be needed. These might then be reviewed by the documentation specialist, in which case the statistical machine learning approach may provide a more desirable starting point for the documentation specialist, with suggestions for the outcome, suggestions for one or more codes, and suggested queries that may be needed

At this point, any actions taken by the documentation specialist with respect to suggestions offered by the statistical machine learning approach may be used to generate an updated or new statistical model (e.g.,, a “confidence” model) based on the relationship between the available clinical and linguistic evidence, the outcome, codes, and/or queries predicted by the original model, and the selections or agreement of the documentation specialist with the predictions of the automated system. Then, the computer may apply the confidence model to determine whether future patient documentation should be given outcome (3) (i.e., sent to a human reviewer when confidence is low) instead of the chosen outcome that might otherwise be proposed.

For both a rules-based approach and a statistical machine learning approach, in cases where outcome (3) occurs (i.e., cases where a human reviewer is needed to complete the coding process), the available evidence may be used to increase the speed with which a human reviewer can complete documentation by identifying and jumping to an intermediate step in the coding process from which a documentation specialist can begin the coding process. In a rules-based approach, identified clinical terms and context may be used to link directly to intermediate code path steps, such as jumping to outcome (2) or outcome (3) based on satisfying one or more rules with respect to the content of a medical record. Similarly, in a statistical/machine learning approach, the system may gather data to train a model that maps linguistic and clinical evidence to a code path, which may then be followed for new patient documents in order to predict the most likely intermediate code path based on available linguistic and clinical evidence.

In many cases, a rules-based approach and a statistical machine learning-based approach may be used together to define a “hybrid” approach. A hybrid rules-based and statistical system may be used on the same documentation, then selection of the outcome, codes, or queries may be based on the output of both approaches. The choice may be made based on rules and or statistics (e.g. for specific rules or queries, the system may choose the rules-based outputs and otherwise, the system may choose the machine learning output). Statistical confidence may also be used, in which case, the system may build a confidence model based on the rules-based system, then compare the confidence of the rules-based outputs to the confidence of machine learning outputs. In this case, the system may choose results having the highest confidence metric, with a first confidence metric being defined for the outcome of a rules-based approach and a second confidence metric being defined for the machine learning approach. In still other cases, a confidence metric may be defined only for the outcome defined by the machine learning approach, and the outcome defined by the rules-based approach may be used as the default approach whenever the confidence metric is below some threshold. In these cases, the outcome defined by the machine learning approach may be used when the confidence metric defined by the machine learning approach exceeds a threshold.

Furthermore, in some hybrid examples, machine learning may be applied to only some of the steps of the coding process, such as that associated with the identification and coding based on key words in the medical record. In this case, the computer may apply a rules-based approach up to the point where key word searching occurs. At the point of key work searching, the computer may adaptively define at least some of the key terms based on previous searches performed by one or more users (i.e., key word searches performed by previous documentation specialists on previously coded documents). The computer may cause the display of possible terms to the one or more users, and may search for possible terms based on selections by the users (i.e., the documentation specialists). Based on such selections, the key terms may be adaptively defined over time. Thereafter, the computer may automatically search for the adaptively defined key terms, e.g., when one of the suppression codes does not appear in the medical record. That is, if the documentation specialist associates key terms or phrases with particular codes or particular actions in the coding process (such as queries to the physician), the computerized system may learn over time, and automate these associations for automated output, or automated suggestions to the documentation specialist with respect to future documents being coded.

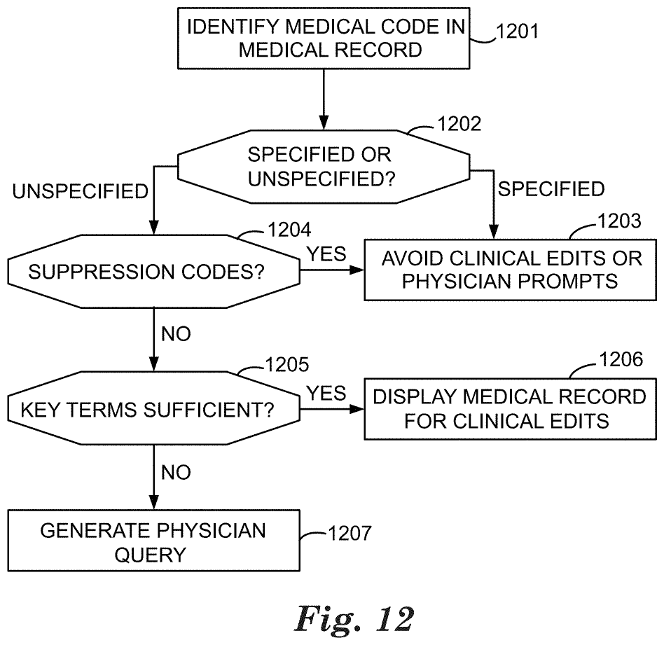

FIG. 12 is a flow diagram illustrating a technique consistent with this disclosure. FIG. 12 will be described from the perspective of computer 110 of FIG. 1, although the system of FIG. 2 or other systems could also be used to perform such techniques. As shown in FIG. 12, editing module 102 identifies a medical code within a medical record stored in the memory 114 (1201). The medical record may be one of many medical records within raw medical data 118 needing review by a documentation specialist. Editing module 102 identifies whether the medical code is one of a plurality of specified medical codes or one of a plurality of unspecified medical codes (1202). The specified medical codes are defined as sufficient to reflect and accurately represent a medical condition to a payer and the unspecified medical codes are defined as requiring additional information for medical condition clarity. The payer typically comprises either a governmental payer, or an insurance company, although the techniques of this disclosure may apply to other payers.

If the medical code is one of the specified medical codes (“specified” 1202), editing module 102 avoids clinical edit options or physician prompts (1203). In other words, if the medical code is specified (“specified” 1202), editing module 102 avoids causing display of clinical edit options via specialist prompts 136 on output device 130 for the medical record, and editing module 102 also avoids the generation of a query for further input by a physician, e.g., avoids generating physician prompts 138 on output device 130.

If the medical code is one of the unspecified medical codes (“unspecified” 1202), editing module 102 determines whether one of a plurality of suppression codes associated with the medical code appears in the medical record stored in raw medical data 118 in memory 114 (1204). If one of the suppression codes appears in the medical record (“yes” 1204), editing module 102 avoids clinical edits or physician prompts (1203). In other words, if a suppression code appears in the medical record (“yes” 1204), editing module 102 avoids causing display of clinical edit options via specialist prompts 136 on output device 130 for the medical record, and editing module 102 also avoids the generation of a query for further input by a physician, e.g., avoids generating physician prompts 138 on output device 130 (1203).

At this point, if one of the suppression codes does not appear in the medical record (“no” 1204), editing module 102 searches for one or more key terms in the medical record (1205). If one or more key terms exist in the medical record (“yes” 1205) and are sufficient to define a code, editing module 102 displays the medical record for clinical edit options by the documentation specialist (1206). In particular, editing module 102 may generate specialist prompts 136 on output device, e.g., causing display of the editing options for the medical record stored in raw medical data 118 of memory 114. Accordingly, when code additions or modifications are received from a documentation specialist, editing module 102 may store an edited version of the medical record in coded medical data 120 within memory 114. On the other hand, if one or more key terms are present in the medical record (“no” 1205) and do not contain sufficient detail as to define a code, editing module 102 generates a physician query (1207). In particular, if the one or more key terms do not exist in the medical record (“no” 1205), editing module 102 generates physician prompts 138 on output device, e.g., causing display or printout of a query for further input by the physician.

As noted above, medical codes within the medical records may comprise codes defined by the ICD, such as ICD-9 codes or ICD-10 codes, although the techniques are not necessarily limited to ICD medical codes and could apply with respect to other types of medical codes. In particular, other medical codes may be used with the techniques of this disclosure, particularly for billing to insurance companies or other non-governmental organizations, which may define their own code system or may adopt that of the ICD. Like the medical codes, the suppression code may also be defined by the ICD, wherein the suppression codes are more specific than the medical codes. According, a given suppression code may override and “suppress” a broader medical code by providing more specific information on a given condition or procedure coded in the medical record.

In some examples, the key terms are pre-defined, and editing module 102 automatically searches for the key terms within the medical record when one of the suppression codes does not appear in the medical record. In other examples, at least some of the associations between key terms and queries may be adaptively defined, in which case machine learning techniques may be used over time to associate key terms with queries to medical records that are made by the documentation specialist. Accordingly, in this case editing module 102 may adaptively define at least some of the key terms based on previous searches for terms performed by one or more users (e.g., other documentation specialists that performed review and edits or similar types of medical records). For example, editing module 102 may cause the display of possible terms to the one or more users (e.g., as specialist prompts 136), and editing module 102 may then search for ones of the possible terms within a medical record based on selections by the one or more users (e.g., user input in response to specialist prompts 136). In this case, one or more of the associations between key terms and queries may be adaptively defined by editing module 102 over time based on the selections of the possible terms by the one or more users. Moreover, once one or more associations of the key terms and queries are adaptively defined over time based on the selections of the possible terms by the one or more users, editing module 102 may be configured to automatically search for the adaptively defined associations between key terms and queries when one of the suppression codes does not appear in the medical record. In this manner, machine learning techniques may be used over time to associate key terms with selections and/or edits to medical records made by documentation specialists. Additional machine learning techniques are also discussed below.

When causing display of the editing options for the medical record, editing module 102 may cause any of a wide variety of specialist prompts 136 to appear on output device 130. In some examples, specialist prompts 136 may display of at least a portion of data from the medical record in raw medical data 118 to allow for edits by a documentation specialist. Once code additions or modifications are made by the documentation specialist, editing module may cause the edited version of the medical record to be stored in memory 114 as coded medical data 120.

When generating a query for further input by the physician, editing module 102 may automatically or manually, through a documentation specialist, generate physician prompts 138. Physician prompts 138 may comprise a physician documentation request that requests additional details for the medical record. As examples, the requested details may pertain to the medical code, the suppression code, or one or more key terms. In this way, physician prompts 138 can be automated, yet limited to situations in which physician input is actually needed. Accordingly, unwanted or unnecessary queries to the physician can be substantially minimized.

As mentioned above, one or more of the techniques and rules described herein may be replaced or supplemented with machine learning techniques based on statistics. In this case, the actions of documentation specialists can be saved or accumulated over time (e.g., as statistics), and the computer may learn and adapt future coding suggestions based on statistics associated with the prior actions of documentation specialists.

As discussed above, in both a rules-based approach or a statistical machine learning approach, it may be desirable to determine one of three outcomes: (1) automatically coding the document (and either sending the document directly to billing or to a human review for final approval), (2) issuing one or more specificity queries back to the physician to improve the documentation, or (3) determining that the automated system was not confident in its prediction and sending the documentation to a human reviewer to choose (1) or (2). It may be desirable, in some cases, to determine a first outcome based on a set of pre-defined rules and determine a second outcome based on adaptive rules defined by statistical machine learning. Then, the computer may select between the first and second outcomes. Confidence metrics may be defined for one or both outcomes and the selection between the first and second outcomes may be based on the one or more confidence metrics.

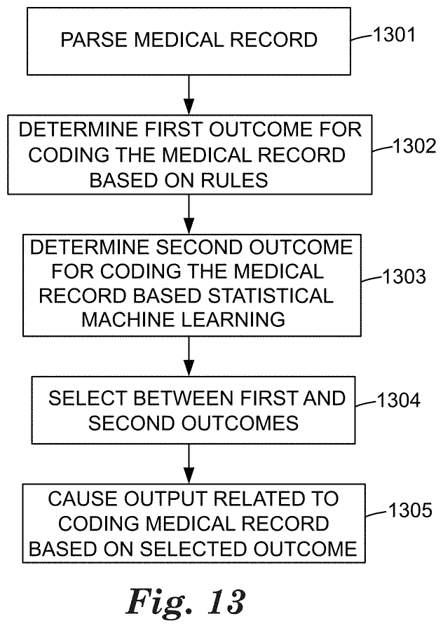

FIG. 13 is a flow diagram illustrating a hybrid technique consistent with this disclosure, which uses both a rules-based approach and a statistical machine learning approach. FIG. 13 will be described from the perspective of computer 110 of FIG. 1, although the system of FIG. 2 or other systems could also be used to perform such techniques. As shown in FIG. 13, editing module 102 editing module parses a medical record stored in raw medical data 118 of memory 114 (1301). Editing module 102 determines a first outcome for coding the medical record based on one or more pre-defined rules (1302). For example, editing module 102 may execute the process of FIG. 12 to determine the first outcome. Editing module 102 also determines a second outcome for coding the medical record based on statistical machine learning (1303). For example, editing module 102 may apply one or more adaptive rules, wherein the adaptive rules are defined based on statistical machine learning based on processing of other medical records. Editing module 102 then selects between the first and second outcomes (1304). In one example, editing module 102 defines a confidence metric associated with the second outcome (i.e., a confidence metric associated with the machine learning outcome), and selects between the first and second outcomes based at least in part on the confidence metric. In another example, editing module 102 defines a first confidence metric associated with the first outcome (i.e., a confidence metric associated with outcome defined by the pre-defined rules-based approach) and a second confidence metric associated with the second outcome (i.e., a confidence metric associated with the machine learning outcome), and selects between the first and second outcomes based at least in part on the first and second confidence metrics. In any case, once editing module 102 selects the outcome, editing module 102 causes output on an output device based on the selected outcome (1305). This may include the generation and output of specialist prompts 136, generation and output of specialist prompts physician prompts 138, or a determination that the coding is complete without the need for any addition input from the physician or the documentation specialist.

The techniques of this disclosure may be implemented in a wide variety of computer devices, such as servers, laptop computers, desktop computers, notebook computers, tablet computers, hand-held computers, smart phones, and the like. Any components, modules or units have been described provided to emphasize functional aspects and does not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

These and other examples are within the scope of the following claims.

1. A method of processing medical data via one or more computers, the method comprising:
identifying, via a processor on a first computer, a medical code within a medical record, the medical record is stored in a memory of the first computer;
determining, via the processor, whether the medical code is one of a plurality of specified medical codes or one of a plurality of unspecified medical codes, wherein the specified medical codes are defined as sufficient to represent a medical condition to a payer and the unspecified medical codes are defined as requiring additional information to represent the medical condition to the payer;
based on determining that the medical code is one of the unspecified medical codes, determining, via the processor, whether one of a plurality of suppression codes associated with the medical code appears in the medical record, wherein a suppression code from the plurality of suppression codes is more specific than the medical code;
based on determining that one of the suppression codes does not appear in the medical record:
searching, via the processor, for one or more key terms regarding the unsupported medical codes in the medical record, wherein the processor also identifies related terms regarding the unsupported medical codes for the one or more key terms as part of the searching;
automatically generating and presenting, via the processor, one or more user interfaces that include one or more clinical edit options, one or more key terms identified by the processor when searching for the one or more key terms, and one or more related terms that the processor identified when searching for one or more key terms, wherein the processor causes the related terms found in the medical record to be presented in the one or more user interfaces with a first visual representation and causes the related terms for which additional information is identified by the processor to be presented in the one or more user interfaces with a second visual representation; and
automatically generating a query for input by a physician that requests additional details regarding one or more of the unspecified medical code, the suppression code, and the one or more key terms.

2. The method of claim 1, wherein the medical code comprises a code defined by the International Classification of Diseases (ICD).

3. The method of claim 2, wherein the suppression code comprises another code defined by the ICD.

4. The method of claim 1, wherein the key terms are pre-defined, the method further comprising automatically searching for the key terms when one of the suppression codes does not appear in the medical record.

5. The method of claim 1, further comprising:
receiving input from the documentation specialist in response to displaying the clinical edit options.

6. The method of claim 5, wherein in response to receiving the input a number of times, the one or more computers adaptively define at least some of associations between:
whether the one or more key terms are present in the medical record; and
the automatically generated query.

7. The method of claim 5, wherein in response to receiving the input a number of times, the one or more computers adaptively define at least some of associations between:
the one or more key terms are not present in the medical record; and
the automatically generated query.

8. The method of claim 1, further comprising:
receiving input from a documentation specialist in response to displaying one or more the clinical edit options, wherein the input modifies one or the medical codes via the first computer.

9. The method of claim 8, further comprising:
upon receiving the input a number of times with respect to the one or more clinical edit options for a particular one of the medical codes or the suppression codes,
automatically generating a recommendation for the documentation specialist with respect to a later-processed medical record.

10. The method of claim 1, wherein based on determining that one of the suppression codes does not appear in the medical record further comprises:
automatically generating, via the processor, a query for display on an output device of a second computer based on the clinical edit options, the one or more key terms, and based on whether the one or more key terms are present in the medical record, wherein the second computer is communicatively coupled to the first computer via a network;
displaying the generated query on the second computer with one or more visual prompts that are configured to receive additional information responsive to the query; and
receiving input from a user of the second computer that is responsive to the displayed query.

11. A computerized system for processing medical data, the system comprising a computer that includes a processor and a memory, wherein the processor is configured to include an editing module, wherein:
the editing module identifies a medical code within a medical record stored in the memory;
the editing module determines whether the medical code is one of a plurality of specified medical codes or one of a plurality of unspecified medical codes, wherein the specified medical codes are defined as sufficient to represent a medical condition to a payer and the unspecified medical codes are defined as requiring additional information to represent the medical condition to the payer;
based on determining that the medical code is one of the unspecified medical codes, the editing module determines whether one of a plurality of suppression codes associated with the medical code appears in the medical record stored in the memory, wherein a suppression code is more specific than the medical code;
based on determining that one of the suppression codes does not appear in the medical record:
the editing module searches for one or more key terms regarding the unsupported medical codes in the medical record stored in the memory, wherein the processor also identifies related terms regarding the unsupported medical codes for the one or more key terms as part of the searching;
the editing module automatically generates and presents one or more user interfaces that include one or more clinical edit options, one or more key terms identified by the processor when searching for the one or more key terms, and one or more related terms that the processor identified when searching for one or more key terms, wherein the editing module causes the related terms found in the medical record to be presented in the one or more user interfaces with a first visual representation and causes the related terms for which additional information is identified by the processor to be presented in the one or more user interfaces with a second visual representation; and
the editing module automatically generates a query for input by a physician that requests additional details regarding one or more of the medical code, the suppression code and the one or more key terms.

12. The system of claim 11, wherein the medical code comprises a code defined by the International Classification of Diseases (ICD), wherein the suppression code comprises another code defined by the ICD, wherein the suppression code is more specific than the medical code.

13. The system of claim 11, wherein the key terms are pre-defined, wherein the editing module automatically searches for the key terms when one of the suppression codes does not appear in the medical record.

14. The system of claim 11, wherein in response to receiving the input a number of times, the editing module adaptively defines at least some of the associations between:
the one or more key terms are present in the medical record; and
the automatically generated query.

15. The system of claim 11, wherein based on determining that one of the suppression codes does not appear in the medical record further comprises:
the editing module automatically generates a query based on the one or more key terms and based on whether the one or more key terms are present in the medical record;
the editing module displays the generated query on the second computer with one or more visual prompts that are configured to receive additional information responsive to the query; and
the editing module receives input from a user of the second computer that is responsive to the displayed query.
